(19) United States
(12) Patent Application Publication
(10) Pub. No.: US 2026/0102218 A1
(43) Pub. Date: Apr. 16, 2026

(54) COVER FASTENING ELEMENT, STERILE COVER FILM, COVER FASTENING SYSTEM, COVER SYSTEM, AND SURGICAL ROBOT WITH COVER

(71) Applicant: B. Braun New Ventures GmbH, Freiburg im Breisgau (DE)

(72) Inventor: Lukas Hahn, Sigmaringen (DE)

(21) Appl. No.: 19/115,904

(22) PCT Filed: Sep. 21, 2023
(86) PCT No.: PCT/EP2023/076029

§ 371 Date: Mar. 27, 2025

(30) Foreign Application Priority Data
Sep. 29, 2022 (DE) 10 2022 125 216.7

(51) Int. Cl. A61B 46/10; A61B 17/00; A61B 34/20; A61B 34/30; A61B 90/00

(52) U.S. Cl. CPC A61B 46/10; A61B 34/20; A61B 34/30; A61B 90/39; A61B 2017/00477; A61B 2034/2055; A61B 2090/3983

(57) ABSTRACT

A medical cover fastening element is configured for releasably fixing a sterile cover film to a navigation marker, preferably to a light emitting diode, in particular to a light emitting diode of a surgical robot. The medical cover fastening element has an opening and a cover fastening interface. The opening extends along a longitudinal axis from an underside to an upper side of the cover fastening element, into which opening a protruding body, preferably a navigation marker, very preferably a light emitting diode, can be inserted from an underside towards the upper side. The cover fastening element can be releasably fastened, force-fittingly and/or form-fittingly, via the cover fastening interface, in particular in the region of the underside of the cover fastening element. A sterile cover film, a cover fastening system, a sterile cover system and a surgical robot can be used with the medical cover fastening element.

### CROSS-REFERENCE TO RELATED APPLICATIONS

This application is the United States national stage entry of International Application No. PCT/EP2023/076029, filed on Sep. 21, 2023, and claims priority to German Application No. 10 2022 125 216.7, filed on Sep. 29, 2022. The contents of International Application No. PCT/EP2023/076029 and German Application No. 10 2022 125 216.7 are incorporated by reference herein in their entireties.

### FIELD

The present disclosure relates to a medical cover fastening element for releasably fixing (temporary coupling) a sterile cover film/cover to a navigation marker, preferably to a light emitting diode as a navigation marker, in particular to a light emitting diode of a surgical robot. In addition, the present disclosure relates to a sterile cover film, a cover fastening system with a navigation marker, in particular an LED as a navigation marker, and a cover fastening element, and moreover relates to a cover system and a surgical robot with a cover.

### BACKGROUND

For tracking a robot, it has a large number of navigation markers, such as a large number of (active or passive) light emitting diodes (LEDs). However, one problem here is that the robot is not sterile and that, for a surgical procedure, a sterile barrier in the form of a radiation-transparent, in particular infrared-transparent, drape/cover film must be inserted. In a particularly simple variant, a sterile transparent cover can be thrown over the robot so that it completely covers the robot and thus provides a sterile barrier to its environment.

However, accuracy studies have shown that the tracking accuracy of covered infrared LEDs decreases significantly due to light reflections at the cover film. Therefore, precise alignment of the transparent sterile drape is of particular importance. Furthermore, safe and easy fastening of the sterile drape currently poses problems.

According to current prior art, there is, for example, a navigated robotic arm with an integrated laser for cutting bone structures. This bone-cutting robot likewise has draped LEDs, with the sterile drape/sterile cover film being attached to the surface of the robot with a contractible, circumferential cord or string. However, a preoperative or intraoperative attachment of the cord (i.e. before or during a surgical procedure/surgery) can be assessed as poor, since covering of the drape and tying up the cord is complex and, in addition, no standardized procedure is provided. Moreover, light reflections of the cover in the immediate vicinity of the LED may lead to further tracking inaccuracies, as there is no high-contrast background.

Moreover, according a prior art document, a microscope head tracking system is being implemented, but this is done using a passive tracking technique by attaching black interfaces on the cover film (i.e. in the sterile area), to which the passive markers can be attached. This configuration has the significant disadvantage that, when the patient is admitted in a non-sterile area, also the microscope head must be tracked. Therefore, both sterile and non-sterile passive markers are required, which entails increased production and sterilization costs and consequently also surgery costs and, moreover, adds another source of error, for example, by accidentally mixing up sterile and non-sterile markers.

Another prior art describes to attach a sterile end effector with integrated LEDs to a non-sterile robot arm. This design has the disadvantage that sterilizable LEDs, correspondingly accompanied by high costs, must be integrated into the end effector and, furthermore, even a power supply for the LEDs through the cover film has to be provided. Another prior art document describes the integration of fixing elements on at least one of the several links of the robot arm to cover LEDs.

### SUMMARY

It is therefore the object of the present disclosure to avoid or at least mitigate the drawbacks of the prior art and to provide a cover fastening element, a sterile cover film/drape, a cover fastening system, a cover system and a surgical robot, which provides particularly easy and secure fastening as well as precise tracking of navigation markers. A further sub-objective is to provide cost-effective fastening of the sterile cover film that is easy to handle.

One basic idea of the present disclosure hence is to use, for fastening a sterile cover film, the navigation markers, in particular the light emitting diodes and, respectively, the LED housings of the light emitting diodes of the robot, in order to dock and undock them in a detachable manner, so to speak. For this purpose, specially designed cover fastening elements are employed that allow precise tracking of navigation markers, in particular light emitting diodes (LEDs), especially infrared LEDs. While the navigation markers such as light emitting diodes (LEDs) themselves do not need to be sterile, a sterile cover film is fastened via the connectable cover fastening element and stretched tightly over the navigation marker, in particular the light emitting diode, thus creating a smooth surface of the cover film and preventing the sterile film from influencing tracking accuracy (light reflections and diffraction). The cover fastening elements (in particular in combination with the LED or an LED housing) can therefore create a smooth surface and significantly improve the tracking accuracy of the navigation marker, in particular of the LEDs, covered with a sterile drape or cover film. The cover film cover fastening elements stretch the sterile cover film tightly over the navigation markers, in particular the tracking LEDs, without however destroying the sterile barrier. This provides a simple, cost-effective, quickly attachable, yet secure and standardized sterile barrier by correspondingly attaching the sterile cover film to the navigation markers, in particular LEDs, of the surgical robot using the special cover fastening elements. The navigation markers (such as LEDs) themselves (or in their housing) are firmly integrated into a non-sterile robot head (as or with an end effector), such as a microscope head or a non-sterile end effector, which is covered accordingly.

In even other words, the technical design has as its basis a sterile cover film, such as an LED cover film, which can be fastened, via the special cover fastening element, through a snap connection or magnetically to a standardized interface, in particular of a navigated unsterile end effector (on or as a robot head), preferably a microscope head. This standardized (LED fastening) interface can also be the housing of the navigation marker, in particular the housing of the (tracking) LED. In addition, the snap lock or magnetic lock prevents unintentional loosening of the cover fastening elements (such as LED covers).

In still other words in accordance with the present disclosure, a medical cover fastening element for releasable fixation of a sterile cover film/ a sterile drape to a navigation marker, in particular a light emitting diode (LED), in particular a light emitting diode of a surgical robot, is provided, comprising: an opening along a longitudinal axis (of the cover fastening element) from an underside to an upper side of the cover fastening element, into which opening a protruding, in particular rotationally symmetrical, body, preferably a navigation marker, very preferably a light emitting diode with a housing, can be inserted in the direction of the upper side, including a cover fastening interface, in particular in the region of the underside of the cover fastening element, via which the cover fastening element can be releasably fastened force-fittingly and/or form-fittingly (configured so as to be connectable and detachable) (with respect to the protruding rotationally symmetrical body, in particular the navigation marker such as, for example, the LED), wherein the cover fastening element is particularly configured so as to be sterile and/or sterilizable.

The term underside defines the side of the cover fastening element that faces the surface to be attached, for example, the surface of the surgical robot or the navigation marker. In contrast, the upper side of the cover fastening element forms the end free side of the cover fastening element, which is oriented outwardly towards the environment.

The navigation marker is used for tracking by a navigation system and, respectively, a tracking system (for example, an optical tracking system) in order to determine at least one position of the navigation marker in space. In one embodiment, the navigation marker can be a traceable LED.

Advantageous embodiments are explained in particular below.

According to a preferred embodiment, the cover fastening element can be formed as a fastening clip with a snap connection as cover fastening interface, in which at least one, in particular a plurality of latching fingers/clip arms extend in the direction of the longitudinal axis towards the underside, and can be elastically deflected in the radial direction to hold the cover fastening element force-fittingly and/or form-fittingly, the latching finger preferably having an end protrusion radially inwards and/or radially outwards for forming an undercut for a form-fit. This radially elastic latching finger thus causes, when deflected radially outwards, elastic prestressing radially inwards and vice versa. In this way, fastening can be achieved through a frictional connection (i.e. a force-fitting connection when the latching finger makes radial contact after being elastically deflected) and/or through a form-fitting connection via a latching protrusion and a latching undercut.

According to a further embodiment, the cover fastening element can have evenly distributed, in particular rotationally symmetrically arranged, latching fingers around its opening, which protrude towards its underside and ensure homogeneous peripheral fixing of the cover fastening element, and in particular the latching fingers themselves form the underside, i.e. the last elements towards the underside. The circumferentially evenly distributed latching fingers can be radially deflected in this way and can snap in circumferentially, for example, through a respective end locking protrusion, in order to fasten the cover fastening element.

In particular, the cover fastening element may comprise, as a cover fastening interface, at least one magnet for a magnetic connection in the region of the underside, in particular an annular magnet concentrically arranged around its longitudinal axis, which preferably forms at least part of the underside of the cover fastening element itself, to attach the cover fastening element to a complementary magnet or to a metallic surface in a force-fitting manner. A magnet thus is firmly integrated in the cover fastening element itself in order to fix the cover fastening element (magnetically) at least force-fittingly.

According to one embodiment of a (separate) magnetic cover fastening element/LED cover, a magnet hence can be integrated permanently in the non-sterile end effector, in particular the microscope head. The cover fastening element, e.g. a plastic part, with an integrated magnet, can be pulled over the LED and the magnetic force creates a detachable, force-fit connection. This tensions the cover film and creates a smooth surface for optimal tracking of the LED.

In particular, the cover fastening element can be made of a thermoplastic material with an annular recess/groove into which the magnet can be inserted.

In particular, the magnet is flush and is merely encompassed radially outside. In this embodiment, the magnet thus is exposed as the underside of the cover fastening element.

Preferably, the cover fastening element, in the region of its upper side, can have at least one, in particular peripheral, radially outward protrusion to form an undercut for manual handling. In this way, the cover fastening element can also be easily removed again manually if necessary.

In particular, the cover fastening element may be rotationally symmetric and may have a conical shape/shaping in the direction of the longitudinal axis, wherein in particular, an outer diameter at the upper side is larger than an outer diameter at the underside to provide good handling, and/or wherein in particular an inner diameter on the upper side is larger than an inner diameter of the underside in order to ensure good visibility of the navigation marker such as the light emitting diode. The conical shape of the (radial) outer surfaces also provides good handling, since this shape results in an undercut. If the radial inner surface, approximately from the height of a radiation plane of the LED, likewise has a conical shape, visibility (when attached to the LED) of the emitted radiation of the LED is improved, since a funnel-shaped design expands the radiation area.

In particular, the cover fastening element may have a conical shape. The conical shape of the cover fastening element, in particular the fastening interface of the cover fastening element, reduces strain on the cover film and in addition allows for easy mounting of the LED cover.

According to a further embodiment, the cover fastening element, in the direction of the longitudinal axis towards its upper side, can have a radially outward step on its radially inner side in order to form, in the region of the upper side, a ring offset and spaced apart from a longitudinal axis, in order to enlarge an emission field in particular for a light-emitting diode. A type or shape of a plate is created, in the center of which the radiation source (covered by a sterile cover film) has a wall further away from it on one side and a radiation angle can be increased accordingly.

Preferably, the cover fastening element itself can be made of or consist entirely of polyoxymethylene (POM; thermoplastic resin).

In particular, fixing or fastening of the sterile cover film can be performed from the sterile side. For this, the sterile cover fastening element can be simply clipped (e.g. by means of a snap lock) onto and/or magnetically attached to the navigation marker, in particular the light-emitting diode, from the sterile side of the sterile cover film. In this configuration, for example, a standard cover film, which forms the sterile barrier, can be placed over a surgical robot including the navigation markers. In order to fix the sterile cover film to the robot (so that it does not fall off) on the one hand, and to ensure a defined surface over the navigation markers, on the other hand, the sterile cover film is attached to the navigation markers from the sterile side using the sterile cover fastening elements. This creates a simple and safe configuration.

Preferably, the cover fastening element can be dark in color, in particular, completely black, in order to absorb reflected light as best as possible and moreover, increase contrast with the navigation marker, in particular the light emitting diode.

In particular, the cover fastening element has a dark color on its surface, in particular black (or is black in color). The black color has the advantage that the infrared LEDs are easier to see due to the higher contrast.

According to one embodiment, the cover fastening element may have a knurled radial exterior to improve manual handling. Specifically, the cover fastening element may have a plurality of circumferential corrugations/recesses (as grooves) spaced apart from each other in longitudinal axis.

In particular, the cover fastening element can be designed as a snap connection and the latching fingers can have a latching protrusion pointing radially outwards in order, when elastically prestressed radially inwards and fastened to, in particular, the LED, to engage in an, in particular, peripheral inner groove of the LED housing.

In particular, the cover fastening element can be designed to be rotationally symmetrical. This means that no special direction of insertion of the cover fastening element is required and a user can place and fasten the cover fastening element in any orientation about its longitudinal axis.

Preferably, the cover fastening element can have only rounded edges so as not to damage the cover.

In particular, when viewed in a longitudinal section along the longitudinal axis, the cover fastening element may have a concave cross-sectional shape or an L-shaped cross-sectional shape, such as in a rotationally symmetrical design.

With regard to a sterile cover film for releasable attachment to a navigation marker, in particular a light emitting diode, in particular attachment to a light emitting diode of a surgical robot, the problem is solved by rigidly fixing/non-releasably attaching/fastening at least one cover fastening element to the sterile cover film according to the present disclosure, and in particular by fixing the cover film to the upper side of the cover fastening element such that the sterile cover film sheet forms the sterile barrier towards the outside.

In addition to a system including a separate sterile cover film and the cover fastening elements the cover fastening elements are integrated into the film itself at the intended positions in this embodiment of the sterile cover film so that a specially adapted sterile cover film for covering, for example, a robot and a cover of the navigation markers, in particular the light emitting diodes, is provided.

In one variant of an integrated magnetic cover fastening element of the sterile cover film, the cover fastening element is firmly integrated on the inside of the cover film. This connection can be produced in particular by gluing, film welding and/or a joining process. By producing the magnetic connection, the film is stretched over the LED and allows the LED to be accurately tracked.

In the embodiment of an integrated snap-fit element and an integrated cover fastening element with snap-fit function, respectively, the cover fastening element can be firmly integrated in the cover film. For fixing the cover fastening element with sterile cover film to the LED fastening interface, the cover fastening element has a snap geometry. In particular, the cover film can be stretched over the tracking LED by connecting the LED fastening interface and the cover fastening element.

With regard to a cover fastening system, the task is solved in that it has a navigation marker, in particular a light emitting diode, and a cover fastening element according to the present disclosure, or a sterile cover film according to the present disclosure, and the navigation marker, in particular the light emitting diode, has an LED fastening interface, the LED fastening interface and the cover fastening interface forming complementary coupling structures that are matched to one another and interact so that the cover fastening element can be fastened to the navigation marker, in particular the light-emitting diode, releasably force-fittingly and/or form-fittingly.

In particular, an LED fastening interface and/or cover fastening interface with undercut can provide a secure fit of the sterile cover fastening element (e.g. LED cover) while also allowing the cover fastening element to be attached and released with manual force. This means that a temporary (secure) fixation of the cover fastening element can be created, which can be manually released again if necessary, but is designed such that it cannot be released unintentionally. In particular, the cover fastening element can be designed such that, for releasing, a force higher than a specified threshold is required in order to prevent unintentional release.

In particular, the cover fastening system can have a rotationally symmetrical funnel housing, into which the housing of the light-emitting diode is inserted centrally.

Preferably, the navigation marker, in particular the light-emitting diode, can have a ring magnet as the LED fastening interface, which interacts with a magnet with opposite poles, in particular a ring magnet of the cover fastening interface, in order to provide a force-fit (complementarily polarized) fastening.

According to one embodiment, the navigation marker, in particular the light-emitting diode, can have a hollow-cylindrical LED housing along an LED longitudinal axis and, as an LED fastening interface, can have a circumferential groove on the radial outside of the LED housing to form a circumferential undercut that interacts with the radially elastic latching fingers with latching protrusions in a radially inward direction of the cover fastening interface.

According to a further embodiment, the cover fastening element can be designed as a fastening clip with a snap connection as the cover fastening interface, wherein a circumferential gap is formed between an LED housing of the navigation marker, in particular the light emitting diode, and the cover fastening element, which is designed so that a sterile cover film is received in it in a precisely fitting manner and in particular, the width of the gap corresponds to the thickness of the cover film or is slightly smaller (e.g. 10% smaller), so that the cover film is clamped between the cover fastening element and the light emitting diode (geometrically fitting).

With regard to a sterile cover system the tasks are solved by: for a surgical robot including a robot arm connected to a robot base and a robot head (as or with an end effector) connected to the robot arm, the robot arm having at least one motorized robot arm link/segment, in particular a plurality of actively actuatable robot arm segments, as well as a plurality of navigation markers, in particular light emitting diodes (LEDs) that are arranged on a surface of the at least one robot arm segment and/or on the robot head, wherein these navigation markers, in particular light emitting diodes, each protrude at least partially with respect to the surface, wherein at least a partial quantity of the navigation markers, in particular the light emitting diodes, in particular all of them, comprises an LED fastening interface, the sterile cover system further comprising: a surgical cover film adapted to be placed over the navigation markers, in particular light emitting diodes, located on the surface on a robot arm segment and/or robot head, and a plurality of cover fastening elements according to the present disclosure that interact with the surgical cover and are designed to be releasably coupled above the navigation markers, in particular light emitting diodes, via their LED fastening interface, wherein each of the cover fastening elements has an opening for the navigation marker, in particular the light emitting diode, into which the navigation marker, in particular the light emitting diode, can be inserted, the cover fastening element having a cover fastening interface complementary to the LED fastening interface, which interacts with the fastening interface to secure a portion of the surgical cover film to the navigation markers, in particular light emitting diodes.

In particular, the sterile cover system can be provided as a set in a sterile package comprising the sterile cover film and the sterile cover fastening elements.

In particular, the cover system can be adapted so that the fixation or attachment by the cover fastening element is performed from a sterile side.

With regard to a surgical robot for a surgical procedure on a patient, the tasks are solved in that the robot comprises: a robot arm attached to a robot base and a robot head (as or with an end effector) connected to the robot arm, wherein the robot arm has at least one motorized robot arm link/segment, in particular a plurality of actively actuatable robot arm segments, a plurality of navigation markers, in particular light emitting diodes (LEDs) that are arranged on a surface of the at least one robot arm segment and/or on the robot head, these navigation markers, for example light emitting diodes, each at least partially protruding with respect to the surface, wherein at least a partial quantity of the navigation markers, in particular light emitting diodes, in particular all of them, each comprises an LED fastening interface, and wherein the surgical robot further comprises: at least one cover fastening element or sterile cover film or cover fastening system or sterile cover system, respectively, according to the present disclosure.

In particular, the cover fastening element has an undercut in the region of its upper side (i.e. at its upper end) radially outwards, which is suitable for manually removing the cover fastening element. This can make it easier to manually grasp the cover fastening element and pull it off, for example The cover fastening element particularly includes eight snap-in latching fingers/clip arms/latching arms.

It may be advantageous to make the LED fastening interface and/or the cover fastening interface black in color so as to increase contrast with the LED.

With regard to a manufacturing process for the sterile cover film the problems are solved in that this comprises the steps of: arranging a cover fastening element on a sterile cover film; and
joining the cover fastening element to the cover film, in particular through adhesion or welding.

All disclosures relating to the cover fastening element according to the present disclosure also apply with regard to the sterile cover film, the cover fastening system, the cover system and the surgical robot of the present disclosure.

### BRIEF DESCRIPTION OF THE DRAWINGS

The present disclosure will be described in more detail below by reference to the accompanying figures, of which:

FIG. 1 is a perspective view of a cover fastening element according to a preferred embodiment for use in a cover fastening system of a preferred embodiment;

FIG. 2 shows a longitudinal section view through the cover fastening system of FIG. 1, with a transparent sterile cover film interposed between the LED and the cover fastening element but not yet attached;

FIG. 3 is a further longitudinal section view as in FIG. 2, but with the cover fastening element attached to the LED and securing the sterile cover film in place;

FIG. 4 is a perspective view of the state of the cover fastening system as shown in FIG. 3;

FIG. 5 is a perspective view of a surgical robot of a preferred embodiment of the present disclosure comprising the cover fastening system and cover fastening element from FIGS. 1 to 4;

FIG. 6 is a longitudinal section view of a cover fastening system of a further preferred embodiment, wherein the cover fastening element of a further preferred embodiment comprises a magnet for fastening to an LED including a magnet, with the cover fastening element not yet attached;

FIG. 7 is another longitudinal view as FIG. 6, only with the difference that the cover fastening element is fastened to the LED and tensions and clamps the sterile cover film;

FIG. 8 is a perspective view of the cover fastening system from FIG. 7;

FIG. 9 is a perspective view of the cover fastening element of FIGS. 6 to 8;

FIG. 10 is a perspective partial view of a surgical robot according to a further preferred embodiment, to the robot head of which the sterile cover film and the cover fastening elements are attached to the LEDs;

FIG. 11 is a partial perspective view of a sterile cover film sheet of another preferred embodiment having firmly integrated cover fastening elements;

FIG. 12 is a longitudinal section view of a cover system of a preferred embodiment including the sterile cover film sheet with integrated cover fastening elements of FIG. 11;

FIG. 13 is another longitudinal section view as FIG. 12, only with the difference that the sterile cover film is attached to the LED via magnets;

FIGS. 14 and 15 show further perspective views of the system from FIGS. 11 to 13;

FIG. 16 is a perspective recess or protrusion of an LED from a surface as a docking area for a cover fastening element according to a further preferred embodiment;

FIGS. 17 and 18 show a longitudinal section view and a perspective view, respectively, of a cover fastening element that interacts with the docking area of FIG. 16 and that has radially outwards latching protrusions;

FIGS. 19 and 20 each show a perspective view of a sterile cover film having integrated cover fastening elements with a snap-fit connection.

The figures are schematic in nature and are only intended to aid understanding of the invention. Identical elements are provided with identical reference signs. The features of the various embodiments can be interchanged.

### DETAILED DESCRIPTION

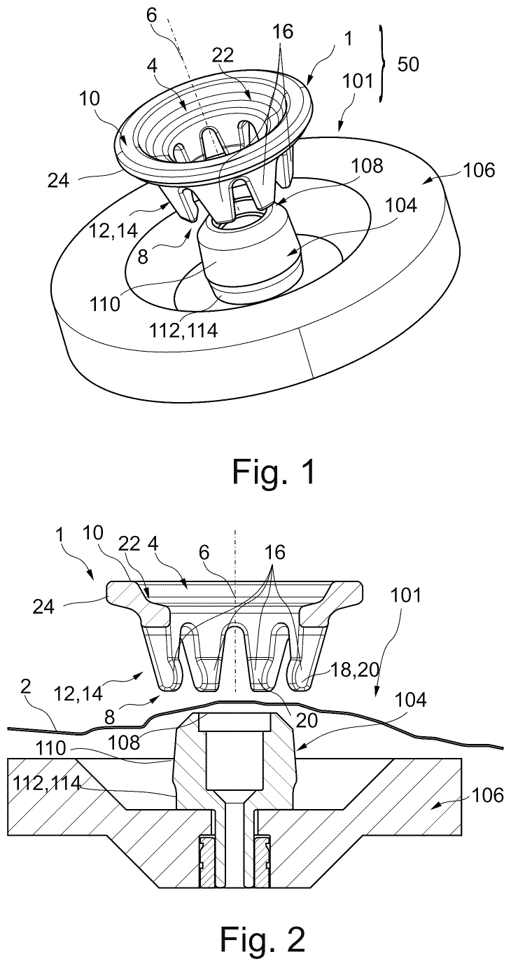

FIGS. 1 to 5 show a cover fastening element, a cover fastening system, a cover system and a surgical robot, each according to a first preferred embodiment of the present disclosure, which interact with each other or form subcomponents of each other.

FIG. 1 shows in a perspective view a medical cover fastening element 1 for releasably securing a sterile cover film 2 (see FIG. 2) to a light emitting diode 101 (as a navigation marker) of a surgical robot 102 (see FIG. 5). As apparent from FIG. 1, the cover fastening element 1 has a central through-opening 4 as a central circular hole, the opening 4 extending along a longitudinal axis 6 between an underside 6 and an upper side 10 of the cover fastening element 1.

A protruding light emitting diode 101 (with LED housing) can be inserted through this opening 4 along the longitudinal axis 6 from the underside 8 towards the upper side 10.

The cover fastening element 1 is formed so as to be sterile and sterilizable for surgical use. In particular, the cover fastening element 1 can have a sterilizable material.

The cover fastening element 1 has a cover fastening interface 12 on or in the area of its underside 8, via which the cover fastening element 1 can be detachably fastened in both a force-fitting and form-fitting manner, as explained below.

Specifically, in this embodiment, the cover fastening interface 12 is designed as a snap connection 14, which has eight freely running and protruding latching fingers 16 along the longitudinal axis towards the underside, which are arranged equally distributed around the circumference.

These latching fingers 16 themselves form the underside 8 of the cover fastening element 1 and are arranged slightly conical, i.e. in the shape of a funnel. As explained later, this facilitates handling and snapping in. Furthermore, the latching fingers 16 are all of the same design and taper in width along the longitudinal axis 6 towards their latching tip 18. In addition, a radially inwardly directed latching protrusion 20 is formed on each latching tip 18.

In the area of the upper side 10, a circumferential step 22 is formed on the radially inner side, which forms an annular collar 24 of the cover fastening element 1 radially outwards. This collar 24 serves to make the cover fastening element 1 particularly easy to handle and moreover serves to expand a possible radiation area.

As further shown in FIG. 1, the (standardized) light emitting diode 101 matching the cover fastening element 1 is equipped with an inner LED housing 104 and a funnel-shaped LED plate 106. Of course, the light emitting diode 101 has a light emitting radiation source (a diode) in the center of the LED housing 104 in order to be tracked.

The LED housing has an essentially cylindrical basic form with a flat LED upper side 108 (from which the radiation is emitted), with a rounded transition from the side wall to the upper side (similar to the head of a Lego figure). In the area of contact between the LED housing 104 and the LED plate 6, a circumferential groove 112 is applied on the radial outer side 110, which forms an undercut for the light-emitting diode 101. As described later in FIGS. 2 to 4, this groove 112 serves for fastening the cover fastening element 1 and forms an LED fastening interface 114 that is complementary to the cover fastening interface 12. The LED plate 106, in turn, has a black surface and favors a contrast to the light-emitting diode 101 and thus tracking. The plate shape also creates a kind of soft transition.

The cover fastening element 1 and the light emitting diode 101 together constitute the cover fastening system 50 of a preferred embodiment of the present disclosure. In this case, the light emitting diode 101 has the LED fastening interface 114, and the LED fastening interface 114 as well as the cover fastening interface 12 match and interact such that they form complementary coupling structures, so that the cover fastening element can be releasably fastened to the light emitting diode in a force-fitting and/or form-fitting manner. The term coupling structure is not only to be seen geometrically, but also includes a functional structure for coupling via a magnet or a magnetizable structure (electromagnet).

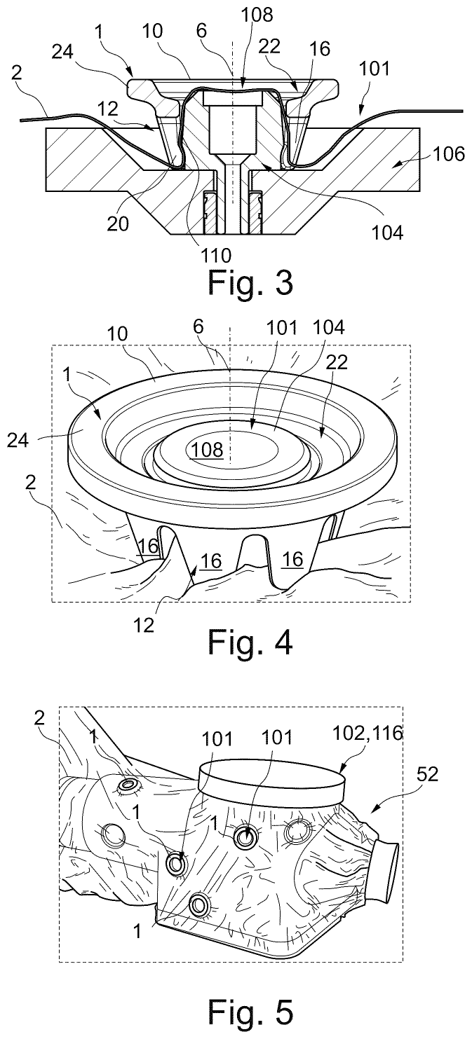

FIG. 2 shows a longitudinal section of the cover fastening element 1 from FIG. 1, but with the sterile cover film/sterile cover drape 2 interposed between the cover fastening element 1 and the light emitting diode 101. As in FIG. 1, FIG. 2 shows the unfastened state. If the cover fastening element 1 is now guided along its longitudinal axis 6 towards its underside 8 to the light emitting diode, the state shown in FIG. 3 results when it stops at the LED plate 6.

FIG. 3 shows the state of the cover fastening system 50 or the cover fastening element 1, in which it is seated in a form-fitting and force-fitting manner on the light-emitting diode 101 and the sterile cover film 2 is clamped between the light emitting diode 101 and cover fastening element 1. When lowering FIG. 2 to FIG. 3, the latching fingers 16 apriori apply the sterile cover film 2 on an upper area of the LED housing 104 and the radially elastic prestressing of the latching fingers 16 stretches the film over the top of the LED 108. When further lowering the cover fastening element 1, the cover film 2 inevitably is pulled downwards through the latching fingers 16 and stretched further over the LED upper side. In the end position of the cover fastening element 1 shown in FIG. 3, all latching fingers 16 homogenously engage in the groove 112 around its circumference and the latching fingers snap back at least partially against their preload in the direction of their static position without the application of force. This results in a form fit and additionally a force fit, and the cover film is held and fastened securely and lies perfectly tensioned over the radiation opening through which the radiation is emitted. By the predefined position of the tensioned cover film 2, reflections can be prevented and a standardized sterile cover for the light-emitting diodes 101 can be created.

The LED plate 106 guides the cover film 2 to the sides over a kind of embankment back to the “normal” surface, where it can continue to cover the surgical robot 102.

FIG. 4 shows a perspective view of the cover fastening system 50 with cover fastening element 1, which tightens the cover sheet 2 tautly over the light emitting diode 101.

FIG. 5 in turn shows a perspective view of the surgical robot 102 of a first preferred embodiment, which has, on its robot head 116, cover fastening elements 1 for fastening the cover film 2 to light emitting diodes 101.

FIGS. 6 to 10 show a further, in each case second preferred embodiment of a cover fastening element 1, a cover fastening system 50, a cover system 52 and a surgical robot 2.

In contrast to the previous embodiment(s), no geometric snap connection is used, but a magnetic coupling and thus attachment.

Specifically, FIG. 6 shows a cover fastening element 1 with an annular magnet 26 on its underside 8. The light emitting diode 101, on the other hand, also has an annular magnet 118. Between the light emitting diode 101 and cover fastening element 1, in the unfastened state, cover film 2 again is located.

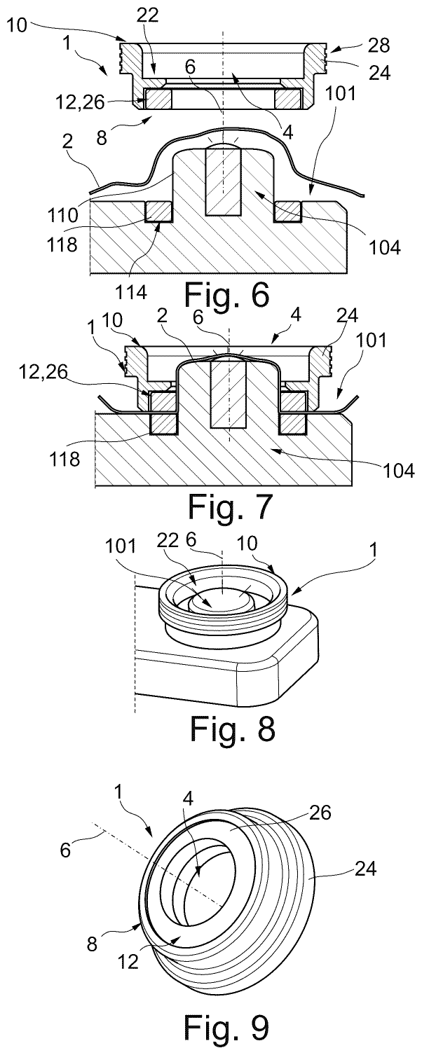

If now, analogous to the procedure of the first embodiment, cover fastening element 1 is lowered, the cover fastening element 1 is fastened to the light emitting diode, as shown in FIG. 7. Specifically, between the radially outer side 110 of the LED housing 8 and the magnet 26, which forms cover fastening interface 12, a gap is provided such that the cover film fits exactly and is pulled downwards by very light press fit. In this way, the cover film 2 is tightened above the radiation source of light emitting diode 101 and forms a sterile barrier. The light emitting diode 101 itself does not have to be sterile.

The cover fastening element 1 has a knurled radial outer structure 28 to make handling, such as pulling the cover fastening element 1 off the light emitting diode 101, easier. Again, a step 22 is formed which enlarges the collar 24 and increases an angle of possible radiation of the radiation source.

FIGS. 8 and 9 show the cover fastening element 1, into which the annular magnet is inserted on its underside. In this embodiment, no LED plate 106 is provided, but a flat surface on which the cover film 2 is placed.

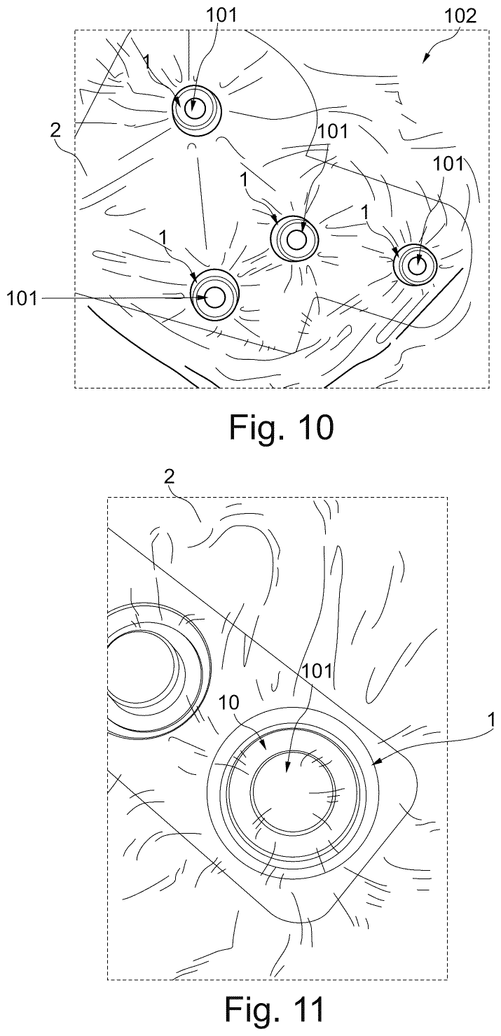

In a perspective partial view FIG. 10 shows a surgical robot 102 of a further preferred embodiment, to which the cover fastening elements 1 of FIGS. 6 to 9 are magnetically held and the cover film 2 is stretched over the radiation source.

FIGS. 11 to 15 show a sterile cover film 2 according to a preferred embodiment, which has integrated cover fastening elements 1. Integrated means that the cover fastening elements 1 are firmly attached to the cover film 2 at predetermined positions, in particular by gluing, film welding and/or another joining method, so that they are rigidly fixed. Through the manufacturing process

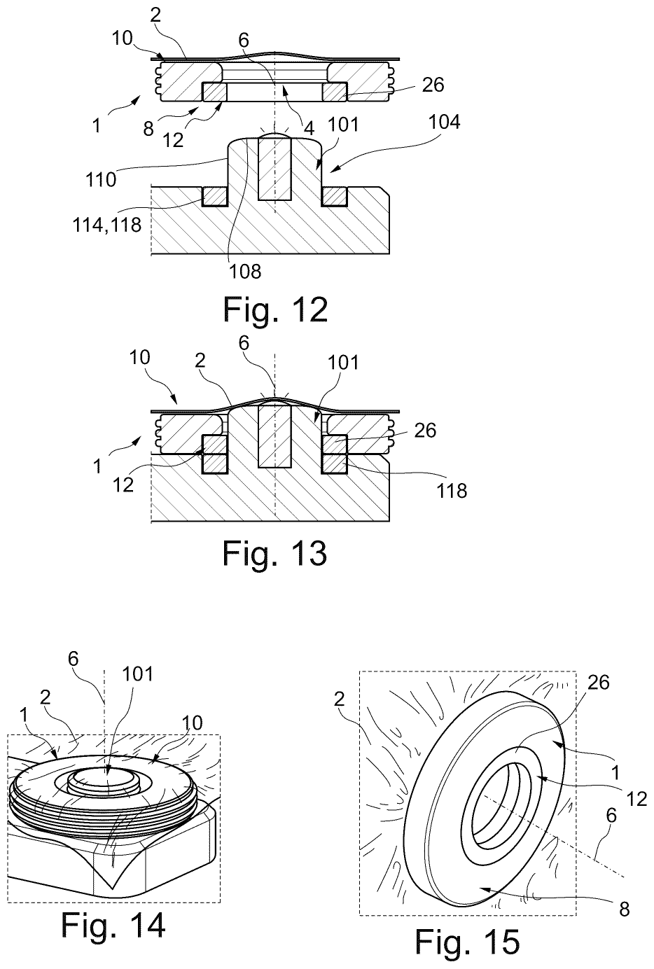

In a longitudinal section view FIG. 12 shows how the cover fastening element 1 joined to the cover film 2 produces a tensioned surface of the cover film 2 in the region of the cover fastening element 1 and, when placed on the light emitting diode 101 as shown in FIG. 13, provides a defined surface of a sterile barrier.

FIGS. 14 and 15 show further views of the sterile cover film 2 with the cover fastening elements 1 (of the cover fastening system 50).

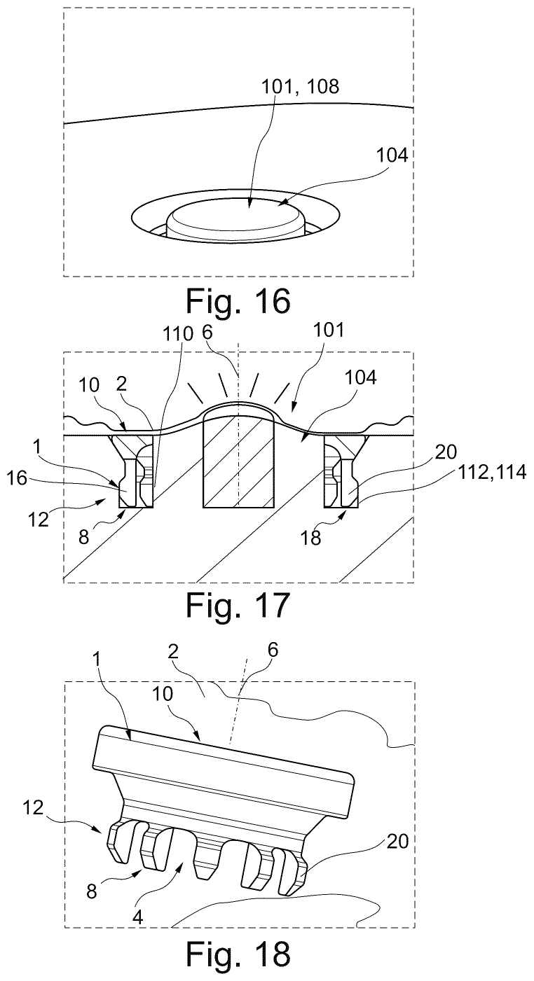

FIGS. 16 to 18 show a further embodiment of a cover fastening element 1, which (similar to the first embodiment) has a snap connection with latching fingers 16 which in this embodiment, however, do not have latching protrusions 20 facing radially inward, but rather facing radially outward and pressing against a radially inner surface of the environment of light emitting diode 101. In this embodiment, a peripheral (inner) groove 112 is provided in a wall around the protruding LED housing 104, which extends concentrically around the LED housing 104. The latching fingers are now prestressed radially inward during insertion and, due to their elastic prestressing force, accordingly snap in radially outwards into the groove 112.

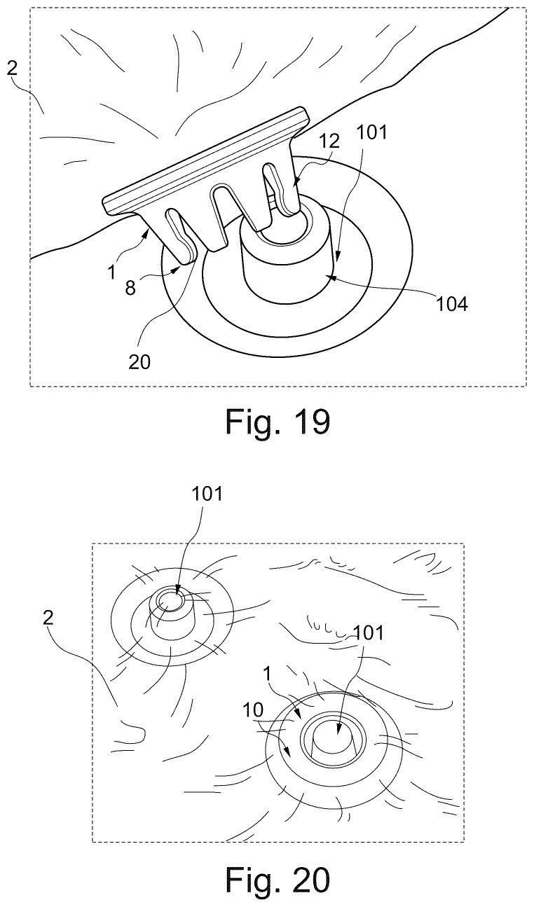

FIGS. 19 and 20 show another preferred embodiment of the sterile cover film 2, which has integrated cover fastening elements 1. In contrast to the first embodiment, the cover fastening elements 1 are configured as snap connections and are essentially the same as the cover fastening element 1 from FIG. 1. The sterile cover film 2 is joined to the cover fastening element 1 at upper side 10, in this case by film welding.

### LIST OF REFERENCE SIGNS

1 Cover fastening element
2 Cover film
4 Opening
6 Longitudinal axis
8 Underside
10 Upper side
12 Cover fastening interface
14 Snap connection
16 Latching finger
18 Latching tip
20 Latching protrusion
22 Step
24 Collar
26 Magnet
28 Knurled (handling) structure
50 Cover fastening system
52 Cover system
101 Light emitting diode/LED (as navigation marker)
102 Surgical robot
104 LED housing
106 LED plate
108 LED upper side
110 Outer side
112 Groove
114 LED fastening interface
116 Robot head
118 Magnet

1.-18. (canceled)

16. A surgical robot for a surgical procedure on a patient, the surgical robot comprising:
a robot arm attached to a robot base and a robot head attached to the robot arm, the robot arm having at least one motorized robot arm segment;
a plurality of navigation markers arranged on a surface of the at least one motorized robot arm segment and/or on the robot head, each of the navigation markers at least partially protruding with respect to the surface, at least one of the navigation markers comprising an LED fastening interface;
a surgical sterile cover film adapted to be placed over the navigation markers; and
a plurality of cover fastening elements interacting with the surgical sterile cover film and being releasably coupleable to and uncoupleable from the navigation markers via the LED fastening interface, each of the cover fastening elements having an opening along a longitudinal axis from an underside to an upper side of the cover fastening element, into which opening one of the navigation markers is introducible, each of the cover fastening elements having a cover fastening interface at an underside of the cover fastening element, the cover fastening interface being complementary to the LED fastening interface, each cover fastening element being releasably fastenable, force-fittingly and/or form-fittingly, via the cover fastening interface, and the cover fastening interface interacting with the LED fastening interface to fasten and secure a part of the surgical sterile cover film to the navigation markers.

17. The surgical robot according to claim 16, wherein each cover fastening element is formed as a fastening clip with a snap connection as the cover fastening interface, in which at least one latching finger extends in a direction of the longitudinal axis towards the underside, and is elastically deflectable in a radial direction to hold the cover fastening element force-fittingly and/or form-fittingly.

18. The surgical robot according to claim 17, wherein the at least one latching finger has an end latching protrusion facing radially inwards for forming an undercut for form fit.

19. The surgical robot according to claim 18, wherein the at least one latching finger comprises a plurality of latching fingers evenly distributed around the opening and protruding towards the underside, the plurality of latching fingers ensuring homogeneous peripheral fixing of the cover fastening element.

20. The surgical robot according to claim 19, wherein each of the navigation markers has an LED housing that is hollow, cylindrical, and extends along an LED longitudinal axis, and a circumferential groove that forms the LED fastening interface on a radial outer side of the LED housing, the circumferential groove forming a circumferential undercut that interacts with the plurality of latching fingers, with the end latching protrusions in a radially inward direction of the cover fastening interface.

21. The surgical robot according to claim 16, wherein each of the cover fastening elements is configured as a fastening clip with a snap connection as a cover fastening interface, wherein a circumferential gap is formed between an LED housing of one of the plurality of navigation markers and the cover fastening element, the circumferential gap configured such that the surgical sterile cover film is accommodated in the circumferential gap with an exact fit.

22. The surgical robot according to claim 16, wherein the surgical sterile cover film is fixed to the upper sides of the cover fastening elements so that the surgical sterile cover film forms a sterile barrier to outside areas.

23. The surgical robot according to claim 16, wherein each of the cover fastening elements has, in a region of the upper side, at least one radially outward protrusion that forms an undercut for manual handling.

24. The surgical robot according to claim 16, wherein each of the cover fastening elements is rotationally symmetrical.

25. The surgical robot according to claim 16, wherein each of the cover fastening elements has, in a direction of the longitudinal axis towards the upper side, on a radially inner side, a step toward a radially outer side to form, in a region of the upper side, a ring offset, the step spaced apart from the longitudinal axis to enlarge an emission field for radiation.

26. The surgical robot according to claim 16, wherein each of the cover fastening elements is formed of polyoxymethylene.

27. A system comprising a sterile cover film and at least one cover fastening element, the system adapted for releasable attachment of the sterile cover film to the navigation markers of the surgical robot according to claim 16,
the at least one cover fastening element having an opening along a longitudinal axis from an underside to an upper side of the at least one cover fastening element, into which opening one of the navigation markers is introducible, wherein the at least one cover fastening element has a cover fastening interface in a region of the underside, wherein the at least one cover fastening element is releasably fastenable, force-fittingly and/or form-fittingly, via the cover fastening interface, and wherein the at least one cover fastening element is fixed to the sterile cover film.
